(19) United States
(12) Patent Application Publication
(10) Pub. No.: US 2014/0349298 A1
(43) Pub. Date: Nov. 27, 2014

(54) PORTABLE, LOW POWER INSTRUMENT FOR THE OPTOELECTRONIC DETECTION OF PATHOGENS USING ISOTHERMAL NUCLEIC ACID AMPLIFICATION PROTOCOLS

(71) Applicants: WILLIAM EUGENE STANCHINA, VALENCIA, PA (US); ABHAY NARAIN VATS, WEXFORD, PA (US); ALEX K. JONES, PITTSBURGH, PA (US); RAGHAV KHANNA, KUTZTOWN, PA (US)

(72) Inventors: WILLIAM EUGENE STANCHINA, VALENCIA, PA (US); ABHAY NARAIN VATS, WEXFORD, PA (US); ALEX K. JONES, PITTSBURGH, PA (US); RAGHAV KHANNA, KUTZTOWN, PA (US)

(21) Appl. No.: 13/548,393

(22) Filed: Jul. 13, 2012

Related U.S. Application Data
(60) Provisional application No. 61/507,785, filed on Jul. 14, 2011.

(51) Int. Cl. C12Q 1/68

(52) U.S. Cl. CPC C12Q 1/686
USPC 435/6.12; 435/287.2

(57) ABSTRACT

This invention relates to a portable, low power apparatus and method for detecting a pathogen of interest in a biological sample obtained from a patient. The apparatus includes a heated pathogen isothermal amplification and diagnostic cell for conducting a loop-mediated isothermal amplification technique. The heated pathogen isothermal amplification and diagnostic cell can include one or more chambers having a corresponding low power heating element connected thereto for heating a reaction mixture contained in the chamber to a desired temperature. Further, the apparatus can include an ultraviolet excitation source, an optical output filter, a photon sensing detector, and an electrical signal detection and amplification circuit to transmit and display output to a wireless device.

### CROSS-REFERENCE TO RELATED PATENT APPLICATIONS

This patent application claims the benefit of provisional U.S. patent application No. 61/507,785, entitled “Portable, Low Power Instrument for the Optoelectronic Detection of Pathogens Using Isothermal Nucleic Acid Amplification Protocols”, filed on Jul. 14, 2011, the contents of which are incorporated herein by reference.

### GOVERNMENT FUNDING

This invention was made with government support under Grant #AI082614 awarded by the United States National Institute of Health (NIH). The government has certain rights in the invention.

### FIELD OF THE INVENTION

This invention relates to an apparatus and method for optoelectronic detection of pathogens in a biological sample using isothermal nucleic acid amplification protocols.

### BACKGROUND OF THE INVENTION

A medical diagnosis of disease in a patient can be provided based on detecting the presence or absence of a pathogen of interest in a biological sample obtained from the patient. The process typically involves collecting the biological sample from the patient, using conventional protocols capable of detecting a particular pathogen of interest, and making a diagnosis based on the presence or absence of the pathogen of interest in the biological sample. Conventional protocols can include using fluorescence labeling, and employing an excitation laser light source and a light sensor system to detect the presence of the pathogen of interest. Due to the limited sensitivity of fluorescence, amplification may be necessary. In measuring fluorescence from a biological sample, only the signal from the pathogen of interest should be amplified.

The instrumentation required to amplify the signal and measure the fluorescence in conventional detection protocols can be very costly and complex. For example, in a laboratory setting, plasmon-controlled fluorescence (PCF) is typically performed on expensive and cumbersome instrumentation, which makes it impractical for use in low resource settings.

There is an emergent demand for point of care (POC) medical diagnosis in low resource settings, such as in developing countries. To address this demand, detection protocols and associated diagnostic instrumentation that is inexpensive, portable, and rapid needs to be developed. This invention satisfies these criteria by employing an amplification technique, e.g., loop-mediated isothermal amplification (LAMP) that is integrated into an inexpensive, portable and simple diagnostic tool which can be used to diagnose infectious diseases, genetic disorder and genetic traits. LAMP is a single tube technique for the amplification of DNA. In LAMP, a target sequence is amplified at a particular temperature using two or three sets of primers and a polymerase. The reaction can be followed by measuring the signals from DNA produced via fluorescent dyes that intercalate or label the DNA.

### SUMMARY OF THE INVENTION

In one aspect, this invention provides a portable, low power apparatus for detecting a pathogen of interest in a biological sample. The apparatus includes a heated pathogen isothermal amplification and diagnostic cell, an ultraviolet excitation source, an optical output filter; a photon sensing detector, and an electrical signal detection and amplification circuit. The heated pathogen isothermal amplification and diagnostic cell includes one or more chambers. Each of the one or more chambers accommodates a reaction mixture and includes a heating element. The reaction mixture includes the biological sample, DNA primer, reaction enzyme, and DNA binding fluorescent dye. The DNA binding fluorescent dye binds to a pathogen of interest to form a fluorescent dye/pathogenic DNA combination. The heating element heats the reaction mixture to a temperature that is sufficient to form the fluorescent dye/pathogenic DNA combination. The apparatus further includes an ultraviolet light excitation source to excite the fluorescent dye/pathogenic DNA combination and to produce a fluorescence light output. An optical output filter is operable to present the fluorescence light output in a predetermined bandwidth of wavelength. A photon sensing detector captures and converts the fluorescence light output to an electrical signal. An electrical signal detection and amplification circuit transmits and displays a measurement of the fluorescence light output to a wireless device.

The measurement of the fluorescent light output in the biological sample can be compared with a measurement of fluorescent light output from a non-pathogen of interest-containing sample, e.g. a control sample, to detect the presence or absence of the pathogen of interest in the biological sample. If the measurement of the fluorescent light output in the biological sample has intensity greater than the measurement of the fluorescent light in the control sample, the presence of the pathogen of interest is detected. If the measurement of the fluorescent light output in the biological sample has intensity comparable with, e.g., not measurably greater than, the measurement of the fluorescent light in the control sample, it is determined that the pathogen of interest is absent or its concentration is below a predetermined detection limit. The detection of the presence or absence of the pathogen of interest in the biological sample can be used to diagnose a medical condition in the patient.

In another aspect, this invention provides a portable, low power method of detecting a pathogen of interest in a biological sample. The method includes obtaining the biological sample from a patient; preparing a reaction mixture including combining the biological sample with DNA primer, reaction enzyme, and DNA binding fluorescent dye; heating the reaction mixture to a temperature sufficient to bind a pathogen of interest to the DNA binding fluorescent dye to form a fluorescent dye/pathogenic DNA combination; exciting the fluorescent dye/pathogenic DNA combination by employing a ultraviolet light excitation source; producing a fluorescent light output; filtering the fluorescent light output using an optical output filter; capturing the fluorescent light output and converting the captured fluorescent light output to an electrical signal using a photon sensing detector; and amplifying and displaying a measurement of the fluorescent light output on a wireless device.

The measurement of the fluorescent light in the biological sample can be compared with a measurement of fluorescent light from a non-pathogen of interest-containing sample, e.g., a control sample, to detect the presence or absence of the pathogen of interest in the biological sample. If the measurement of the fluorescent light in the biological sample is greater than the measurement of the fluorescent light in the control sample, the presence of the pathogen of interest is detected. If the measurement of the fluorescent light in the biological sample is comparable with the measurement of the fluorescent light in the control sample, it is determined that the pathogen of interest is absent or its concentration is below a predetermined detection limit. The detection of the presence or absence of the pathogen of interest in the biological sample can be used to diagnose a medical condition in the patient.

In still another aspect, this invention provides a portable, low power method of diagnosing a medical condition in a patient, including testing two or more biological samples according to the above method and comparing fluorescent light output produced by each of the samples, wherein a pathogen of interest-containing sample has a fluorescent light output of greater intensity as compared to a non-pathogen of interest-containing sample.

### BRIEF DESCRIPTION OF THE DRAWINGS

The invention as set forth in the claims will become more apparent from the following detailed description of certain preferred practices thereof illustrated, by way of example only, and the accompanying drawings wherein;

FIG. 1A shows a plot of spectral output, i.e., light intensity vs. wavelength, measured by a spectrophotometer, of an ultraviolet (UV) light-emitting diode (LED) and amplified loop-mediated isothermal amplification (LAMP) sample prior to the introduction of a filter. FIG. 1B shows a plot of spectral output, i.e., light intensity vs. wavelength, measured by a spectrophotometer, of an UV LED and amplified LAMP sample following the introduction of a filter in accordance with certain embodiments of the invention.

FIGS. 2A and 2B show a heater block in accordance with certain embodiments of the invention. FIG. 2A shows a plan view of a top surface of the heater block. FIG. 2B shows a plan view of a bottom surface of the heater block.

### DETAILED DESCRIPTION OF THE INVENTION

This invention relates to an apparatus and method for optoelectronic detection of pathogens in a biological sample using isothermal nucleic acid amplification protocols. The invention employs an isothermal DNA amplification technique, such as but not limited to, loop-mediated isothermal amplification (LAMP). In certain embodiments of the invention, the presence or absence of a pathogen of interest is detected in a biological sample obtained from a patient by optical excitation and detection of amplified pathogen-specific nucleic acids. A medical diagnosis of disease in the patient can be provided based on the presence or absence of the pathogen of interest in the biological sample. The pathogen of interest can include a wide variety of known pathogens, such as but not limited to, e coli, chlamydia and staphylococcus.

In this invention, a fluorescence light output is used to detect and indicate the presence or absence of the pathogen of interest. The fluorescence light output intensity increases measurably for a sample which contains the pathogen of interest (i.e., positive sample) in comparison with a negative sample. Thus, the fluorescence light output of a biological sample obtained from a patient can be compared with the fluorescence light output of a non-pathogen of interest-containing sample, e.g., a control sample, to detect in a differential manner the presence or absence of the pathogen of interest. When the intensity of the fluorescence light output of the biological sample obtained from the patient is greater than the intensity of the fluorescence light output from the control sample, the presence of the pathogen of interest is detected in the biological sample. The amount or degree of greater intensity (e.g., how much greater is the intensity) in the test sample can depend on the dilution of the pathogen, which can be quantified by conventional calibration tests. When the intensity of the fluorescence light output of the biological sample obtained from the patient is comparable with, e.g., not measurably greater than, the intensity of the fluorescence light output from the control sample, either the absence of the pathogen of interest is detected in the biological sample or it is determined that the concentration of the pathogen of interest in the biological sample is below a predetermined detection limit. The presence of the pathogen of interest can be used to diagnose at least one of the following medical conditions in a patient: infectious disease, genetic disorder and genetic trait.

This invention relates to an apparatus and method that uses portable, low-cost, and/or low power optoelectronic components to excite pathogen-bonded fluorescent dyes in a test sample. In certain embodiments in accordance with the invention, a reaction mixture containing a pathogen of interest and DNA binding fluorescent dye is heated (e.g., employing a heating element) to a temperature which is sufficient to bind the pathogen of interest and the DNA binding fluorescent dye to form a fluorescent dye/pathogenic DNA combination. An excitation source, such as an ultraviolet light source, is employed. Absorption of the ultraviolet light by the fluorescent dye/pathogenic DNA combination causes a positive test sample (e.g., a pathogen of interest-containing biological sample) to fluoresce at longer wavelengths, which are detected by optoelectronic sensors with appropriate electronic circuitry. In certain embodiments, an optical output filter presents the fluorescence in a predetermined bandwidth of wavelength and a photon sensing detector captures, and converts the fluorescence to an electrical signal. An electrical signal detection and amplification circuit can transmit and display a measurement of the fluorescence to a wireless device, such as a cell phone or a palm-top computer. In certain embodiments, the output of the sensor system can be a set of voltage measurements regularly sampled over time for both a positive pathogen sample and a negative (non-pathogen-containing) sample.

The optoelectronic exciter and sensor components can be integrated into test blocks that hold liquid test samples. Further, the test blocks can include heater elements to controllably warm the test samples to predetermined temperatures which are sufficient for amplification and/or multiplication of the test samples.

In certain embodiments, the apparatus and method of the present invention employ commercial off-the-shelf (COTS) optical components. In further embodiments, the low power, COTS optoelectronic components can include battery-operated components.

The method includes obtaining the biological sample from a patient; preparing a reaction mixture including combining the biological sample with DNA primer, reaction enzyme, and DNA binding fluorescent dye; heating the reaction mixture to a temperature sufficient to bind a pathogen of interest to the DNA binding fluorescent dye to form a fluorescent dye/pathogenic DNA combination; exciting the fluorescent dye/pathogenic DNA combination by employing a ultraviolet light excitation source; producing a fluorescent light output; filtering the fluorescent light output using an optical output filter; capturing the fluorescent light output and converting the captured fluorescent light output to an electrical signal using a photon sensing detector; and amplifying and displaying a measurement of the fluorescent light output on a wireless device. Since the fluorescence light output intensity increases measurably for positive pathogen-containing samples in comparison with negative samples, e.g., non-pathogen-containing samples, there is provided a clear indication of when a pathogen of interest is present in a biological sample obtained from a patient.

The biological sample can be obtained from the patient using a variety of conventional techniques and the technique employed is not critical to the invention.

The biological sample obtained from the patient is used to prepare a reaction mixture. The biological sample may contain a pathogen of interest (i.e., a positive sample) or the pathogen of interest may be absent from the biological sample (i.e., a negative sample). The reaction mixture includes, in addition to the biological sample obtained from a patient, DNA primer, DNA binding fluorescent dye and reaction enzyme. The DNA primer provides amplification of the pathogen of interest. The selection of the DNA primer is dependent on the pathogen of interest. The specific DNA primer is selected such that it is unique to the pathogen of interest. The specific primers unique to the pathogen of interest exponentially amplify (replicate) its DNA while, for example, a negative sample remains unamplified. The DNA binding fluorescent dye is selected such that it is capable of binding to the pathogen of interest in the biological sample. The reaction enzyme is selected such that it is appropriate for use with the particular pathogen of interest. The reaction mixture can also include optional components, such as but not limited to, Bst DNA polymerase, dNTPs, and the like. The order of combining these components to form the reaction mixture is not critical to the invention, provided that the DNA binding fluorescent dye is added to the reaction mixture prior to completion of the reaction. In certain embodiments, all of the components of the reaction mixture, including the DNA binding fluorescent dye, can be combined at about the same time. In other embodiments, the DNA binding fluorescent dye can be added at a time after the other components are combined but prior to completion of the reaction.

Heating the reaction mixture to a sufficient temperature causes the DNA primer to amplify (replicate) any pathogenic DNA that may exist in the biological sample. The reaction mixture is heated at a desired or predetermined temperature for a period of time and the particular temperature or temperature range can vary based on the particular components selected for the mixture. The temperature selected is such that it is sufficient to heat the reaction mixture to cause amplification and/or multiplication of pathogenic DNA in the biological sample, and to result in binding of the pathogenic DNA and fluorescent dye to form a fluorescent dye/pathogenic DNA combination. In certain embodiments in accordance with the invention, the reaction mixture is heated to a temperature of about 65° C. over a period of about 30 to about 60 minutes.

An excitation source is used to excite the bound fluorescent dye/pathogenic DNA combination resulting in a fluorescence light output of the reaction mixture. In certain embodiments, the excitation source is an ultraviolet light source, such as a light-emitting diode (LED). In these embodiments, the bound fluorescent dye/pathogenic DNA combination is excited by absorption of ultraviolet light. The wavelength of the LED can vary. In certain embodiments, the LED can have a wavelength of about 370 nm, which results in fluorescence of the reaction mixture in the green spectrum (e.g., 520 nm). Positive test samples fluoresce at longer wavelengths than negative samples.

The fluorescence light output produced by the reaction mixture can be filtered using an optical output filter. In certain embodiments, a bandpass, e.g., narrow bandpass, optical output filter is employed whereby the filter is matched to the output wavelength of the fluorescent dye in the reaction mixture. The filter is effective to diminish or preclude the light output that is not encompassed by the bandpass or bandwidth, e.g., narrow bandpass or narrow bandwidth, of the filter. In certain embodiments, a narrow bandpass optical output filter is employed to present the fluorescence light output in a predetermined bandwidth of wavelength and to reject stray light from the source and/or surrounding environment.

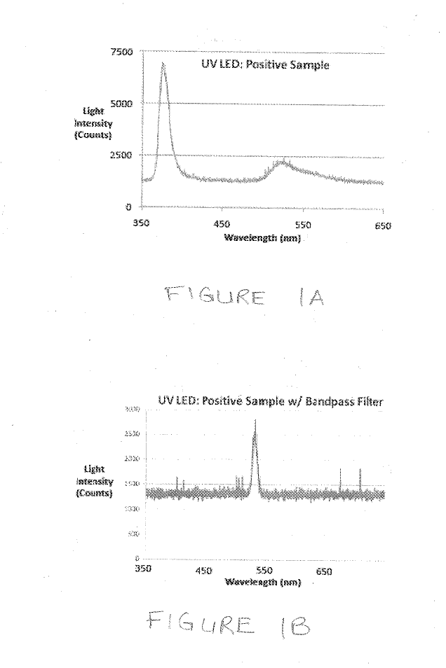

FIGS. 1A and 1B show the spectral output, e.g., light intensity vs. wavelength, of an amplified LAMP sample under UV LED excitation. The spectral output was measured using a spectrophotometer. FIG. 1A shows the spectral output before the introduction of a bandpass filter. FIG. 1B shows the spectral output following the introduction of a bandpass filter. The ultraviolet light is rejected after the filter is engaged. The fluorescent wavelength which yielded maximum light intensity before the bandpass filter was introduced (i.e., 520 nm, 2500 photonic units) was retained after introducing the bandpass filter to the system.

A photon sensing detector is used to capture the fluorescence produced by the reaction mixture. In certain embodiments, the photon sensing detector includes a photoresistor. The photoresistor can detect fluorescence which passed through the bandpass filter.

In certain embodiments, a bandpass filter is placed between an amplified LAMP sample and photoresistor in a test circuit. When the biological sample is excited by the ultraviolet LED, the bandpass filter rejects the ultraviolet light and other stray light, and only accepts the fluorescent wavelength which can alleviate the potential for false positive outcomes.

In other embodiments, the photoresistor can be incorporated in an electrical signal detection and amplification circuit. The circuit can produce an electrical output, such as voltage, that is proportional to the fluorescence light output. The electrical output can be sampled and stored, e.g., digitally, in a non-volatile flash memory.

The data or fluorescence light output obtained from the reaction mixture can be shown or demonstrated in various forms and configurations. In certain embodiments, the data is shown as voltage plotted as a function of time through various output means, such as but not limited to, manual data-taking and graphing or data collection, and graphical display on a personal or laptop computer.

The electrical output of the photoresistor detection circuit can be transmitted to a wireless device. For example, the test results can be transmitted and displayed on a personal mobile wireless device, such as a smart phone (e.g., an iPhone) or other personal digital assistant (PDA). Further, analog-to-digital functions with digital memory for accessing and storing the output data can be included. This data then can be read by a wireless communication system (e.g., Wi-Fi, 802.11) that transmits the data to the personal mobile wireless device. The personal mobile wireless device can serve as a remote portable terminal from which a health care professional accesses and displays the test data for evaluation. The remote device can perform at least one of the following functions: normalize the samples for easy comparison, detect amplification slopes, and transmit the results to other computing devices, including work stations, for further analysis.

The remote device can include a variety of known devices, such as a portable device, for example, a cell phone or a palm-top computer.

In certain embodiments, the LAMP device can be employed in a remote setting where the user can be a finite distance from the LAMP device while still being able to monitor its output using the portable, remote device. If the display device is out of range, the data can be buffered locally in the instrument and transmitted once the device is within an acceptable range.

A medical diagnosis can be made by viewing the graphical data on the remote device.

For positive samples, e.g., pathogen-containing samples, the exponentially replicated pathogenic DNA will bind to the fluorescent dye, which will result in exponentially increasing fluorescence upon ultraviolet excitation. This will be reflected in measuring the response of the photoresistor over time and viewing it on the remote device.

For negative samples, e.g., pathogen-containing samples, the photo-resistor response will remain relatively constant over time, or at least increase much more slowly than a positive sample.

A positive sample shows exponential amplification as compared to a negative sample and therefore, by comparing the time-dependent response of the photoresistor measuring the fluorescence from an unknown, biological sample obtained from a patient with that of a negative control sample or a positive control sample, or both, a diagnosis can be made as to the presence or absence of the pathogen of interest in the biological sample obtained from the patient.

The LAMP technique can be carried out in a wide variety of cells or vessels. For example, the reaction mixture can be introduced into a heated pathogen isothermal amplification and diagnostic cell. In certain embodiments in accordance with the invention, the heated pathogen isothermal amplification and diagnostic cell can include a heater block having one or more chambers or cells formed therein. The chambers or cells being sized and shaped to accommodate or hold a liquid test sample. The heater block can include one or more heating elements. The one or more heating elements can correspond to the one or more chambers or cells such that each of the chambers or cells has a heating element associated therewith. The heating element can include a variety of low power heat sources. In certain embodiments, the heating element is a Peltier heater. In further embodiments, a potentiometer can be connected to the heater block to control the power dissipated by the heating element(s), e.g., Peltier heater(s), such that each sample is heated to the desired or preselected temperature, e.g., about 65° C.

In certain embodiments, wherein the heater block is employed for the LAMP technique, optical channels can traverse the heater block such that an ultraviolet LED can excite the fluorescent dye/DNA pathogenic combinations in the reaction mixture which is contained in each chamber or cell of the heater block. In certain embodiments, a similar optical channel guides the fluorescent light toward an optical detector for each chamber in a direction orthogonal to the incident ultraviolet light.

In certain embodiments in accordance with the invention, all components of the apparatus, with the exception of the remote display device, can be powered using a low power source. For example, the low power source can include the use of one or more batteries. In certain embodiments, two 9-volt batteries can be used. It has been found that the total power consumed by the LAMP device according to the invention can be about 5 watts. In these embodiments, the heating of the reaction mixture can be carried out using simple resistive heating. This resistive heating has been shown to be operable. However, without intending to be bound by any particular theory, it is believed that resistive heating results in slow heating of the samples which may be inconvenient in particular situations. Thus, in other embodiments, Peltier heaters are utilized, as above-described.

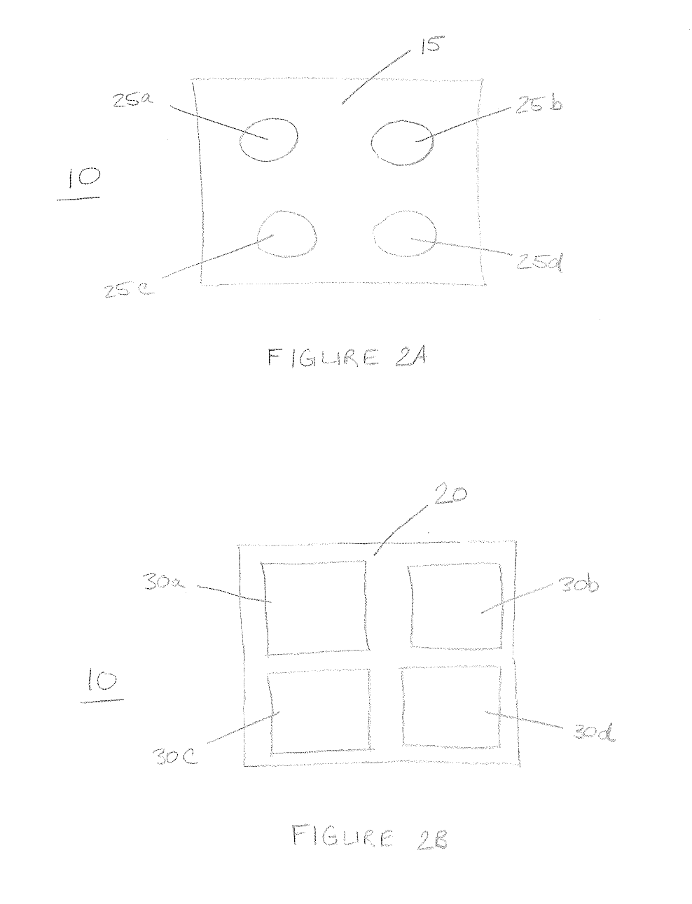

FIGS. 2A and 2B show a heater block 10 in accordance with certain embodiments of this invention. The heater block 10 is shown having a square shape. However, it is understood that the heater block 10 can include a wide variety of shapes and sizes. FIG. 2A shows a top surface 15 of the heater block 10 and FIG. 2B shows a bottom surface 20 of the heater block 10. In FIG. 2A, the top surface 15 is shown having four chambers 25a, 25b, 25c and 25d formed therein. It is understood that the top surface 15 can include one or a plurality of chambers. Each of the four chambers 25a, 25b, 25c and 25d is shown having a round shape. However, it is understood that each of the four chambers 25a, 25b, 25c and 25d can include a wide variety of shapes and sizes. Each of the four chambers 25a, 25b, 25c and 25d can accommodate a reaction mixture (not shown). The reaction mixture can include a biological sample, DNA primer, fluorescent dye, reaction enzyme, and optional components. Optical and opto-electronic components (not shown) can be mounted on the sides of the heater block 10. In certain embodiments, incident ultraviolet light channels and exiting fluorescence light channels (not shown) can be orthogonal to each other and to their corresponding chambers (25a, 25b, 25c, and 25d). For example, in certain embodiments, a photon sensing detector and UV source are arranged othogonally to each other for each sample holder 25a, 25b, 25c and 25d as shown in FIG. 2A.

In FIG. 2B, the bottom surface 20 of the heater block 10 shows four Peltier heaters 30a, 30b, 30c and 30d which correspond to the four chambers 25a, 25b, 25c, and 25d (in FIG. 2A). Each of the Peltier heaters 30a, 30b, 30c and 30d is operable to heat the reaction mixture (not shown) in each of the four chambers 25a, 25b, 25c and 25d to a temperature of approximately 65° C. It is understood that the number of chambers and corresponding Peltier heaters can vary. It is further understood that each chamber may or may not include a corresponding Peltier heater.

In certain embodiments, the apparatus and method of this invention can be used to detect E coli DNA amplified with primers directed against malB gene.

The apparatus of this invention provides at least one of the following benefits over known devices:
An instrument that automatically demonstrates the presence of a pathogen resulting from the isothermal nucleic acid protocols;
A low cost, portable point-of-care instrument for pathogen identification that is appropriate for use world-wide including under-developed countries;
A point-of-care instrument that will enable pathogen identification in a short period of time (e.g., less than about 30 minutes);
A device that does not require on-board computation in a diagnostic instrument by leveraging external portable computation available in devices, such as PDAs and smart phones.

Whereas particular embodiments of the invention have been described herein for purposes of illustration, it will be evident to those skilled in the art that numerous variations of the details may be made without departing from the invention as set forth below:

We claim:

1. A portable, low power apparatus for detecting a pathogen of interest in a biological sample, comprising:
a heated pathogen isothermal amplification and diagnostic cell comprising one or more chambers structured to accommodate a reaction mixture and a heating element, the reaction mixture comprising the biological sample, a DNA primer, a reaction enzyme, and a DNA binding fluorescent dye, wherein the heating element heats the reaction mixture to a temperature sufficient to bind the pathogen of interest and DNA binding fluorescent dye to form a fluorescent dye/pathogenic DNA combination;
an excitation source to excite the fluorescent dye/pathogenic DNA combination and to produce a fluorescence light output;
an optical output filter operable to present the fluorescence light output in a predetermined bandwidth of wavelength;
a photon sensing detector to capture the fluorescence light output and convert said fluorescent light output to an electrical signal; and
an electrical signal detection and amplification circuit to transmit and display a measurement of the fluorescence light output to a wireless device.

2. The apparatus of claim 1, wherein the heated pathogen isothermal amplification and diagnostic cell comprises a heater block having a top surface and a bottom surface wherein the one or more chambers are formed in the top surface, and the heating element comprises one or more heaters connected to the bottom surface to correspond with each of the one or more chambers formed in the top surface.

3. The apparatus of claim 1, wherein the ultraviolet excitation source comprises an ultraviolet light emitting diode.

4. The apparatus of claim 1, wherein the photon sensing detector comprises a photoresistor.

5. The apparatus of claim 1, wherein the wireless device is selected from the group consisting of a personal mobile wireless device and a remote wireless device.

6. The apparatus of claim 1, wherein the personal mobile wireless device is selected from the group consisting of a smart phone and a personal digital assistant.

7. The apparatus of claim 1, wherein the photon sensing detector is incorporated into the electrical signal detection and amplification circuit.

8. The apparatus of claim 1, further comprising a potentiometer connected to the heat source to control the power dissipated therefrom to maintain a temperature of about 65° C.

9. The apparatus of claim 1, wherein one or more of the heated pathogen isothermal amplification and diagnostic cell, ultraviolet excitation source, optical output filter, photon sensing detector, and electrical signal detection and amplification circuit is powered using a low power source.

10. The apparatus of claim 9, wherein the low power source comprises one or more batteries.

11. The apparatus of claim 1, wherein the bandwidth of the optical output filter is matched to an output wavelength of the fluorescent dye in the reaction mixture.

12. The apparatus of claim 1, further comprising comparing an intensity of the fluorescence light output with an intensity of the fluorescent light output of a non-pathogen containing sample to detect presence or absence of the pathogen of interest and employing the presence or absence of the pathogen of interest make a medical diagnosis of the patient.

13. A portable, low power method of detecting a pathogen of interest in a biological sample comprising:
obtaining a biological sample from a patient;
preparing a reaction mixture comprising combining the biological sample with DNA primer, reaction enzyme, and DNA binding fluorescent dye;
heating the reaction mixture to a temperature sufficient to amplify pathogenic DNA present in the reaction mixture and to bind the pathogen of interest to the DNA binding fluorescent dye to form a fluorescent dye/pathogenic DNA combination;
exciting the fluorescent dye/pathogenic DNA combination by employing a ultraviolet light excitation source;
producing a fluorescent light output;
filtering the fluorescent light output using an optical output filter;
capturing the fluorescent light output using a photon sensing detector; and
displaying or storing the fluorescent light output on a wireless device.

14. The method of claim 13, further comprising heating the reaction mixture to a temperature of 65° C.

15. The method of claim 13, further comprising comparing an intensity of the fluorescence light output with an intensity of the fluorescent light output of a non-pathogen containing sample to detect presence or absence of the pathogen of interest and employing the presence or absence of the pathogen of interest make a medical diagnosis of the patient.

16. The method of claim 13, further comprising measuring the fluorescence light output and transmitting an electrical signal arising from the measurement of the fluorescence light output from the wireless device to a remote computing system.

17. The method of claim 13, wherein the wireless device can perform functions selected from the group consisting of normalizing a plurality of biological pathogen-containing samples for comparison, detecting amplification slopes, transmitting the results to other computing devices, and combinations thereof.

18. The method of 13, wherein filtering the fluorescence light output using an optical output filter comprises matching a bandwidth of the optical output filter to an output wavelength range of the fluorescent dye in the reaction mixture.

19. A portable, low power apparatus for diagnosing a medical condition in a patient, comprising:
detecting a pathogen of interest in a biological sample according to the method of claim 13;
comparing intensity of the fluorescence light output produced by the biological sample with intensity of a fluorescence light output produced by a non-pathogen of interest-containing control sample;
indicating the presence of the pathogen of interest in the biological sample wherein the intensity of the fluorescence light output produced by the biological sample is measurably greater than the intensity of the fluorescence light output produced by the non-pathogen of interest-containing control sample; and
indicating the absence of the pathogen of interest or a concentration of the pathogen of interest below a predetermined detection limit in the biological sample wherein the intensity of the fluorescence light output produced by the biological sample is not measurably greater than the intensity of the fluorescence light output produced by the non-pathogen of interest-containing control sample.

20. The method of claim 19, wherein the presence of the pathogen of interest in the biological sample results in diagnosing the patient with a medical condition selected from the group consisting of infectious disease and genetic disorder in the patient.
